(19) United States
(12) Patent Application Publication
(10) Pub. No.: US 2020/0390513 A1
(43) Pub. Date: Dec. 17, 2020

(54) SKIN AND TISSUE PROTECTION DEVICE FOR AESTHETICS TREATMENTS WITH COLD TEMPERATURES

(71) Applicant: HIGH TECHNOLOGY PRODUCTS, S.L., Barcelona (ES)

(72) Inventor: Jose Antonio Sanchez Jaime, Barcelona (ES)

(73) Assignee: HIGH TECHNOLOGY PRODUCTS, S.L., Barcelona (ES)

(21) Appl. No.: 16/975,423

(22) PCT Filed: Mar. 13, 2018
(86) PCT No.: PCT/ES2018/070185

§ 371 Date: Aug. 25, 2020

(30) Foreign Application Priority Data
Feb. 26, 2018 (ES) PCT/ES2018/070145

(51) Int. Cl. A61B 90/00; A61F 7/02

(52) U.S. Cl. CPC A61B 90/04; A61F 7/02; A61B 2090/0463; A61F 2007/0288; A61F 2007/026; A61F 2007/0219; A61B 2090/0427

(57) ABSTRACT

It comprises at least one first layer (1), at least one second layer (2) on said first layer (1) and joined to it (1), defining inner surfaces (3) between both layers which form at least one housing (111), with both layers (1, 2) being porous and absorbent, and at least one cold conducting anti-freezing gel (4), spread in housing (111), all over inner surfaces (3), with a freezing point below −12° C., and with a viscosity of between 2000 and 90000 cPs at 21° C., which is enough to pass through the pores in layers (1, 2) and come out of housing (111), characterized in that it comprises a reinforcement (100) in at least one of its layers (1, 2) such as a stiffening structure.

Skin and tissue protection device for cold aesthetics treatments, of the type comprising at least one first layer, at least one second layer on said first layer and joined to it, defining inner surfaces between both layers which form at least one housing, with both layers being porous and absorbent, and at least one cold conducting anti-freezing gel, spread in the housing, all over the inner surfaces, with a freezing point lower than −12° C., and with a viscosity of between 2000-90000 cPs at 21° C., which is enough to pass through the pores in the layers and come out of the housing; characterized in that it comprises a reinforcement in at least one of its layers, such as a stiffening structure.

### BACKGROUND TO THE INVENTION

Various devices are known in the state of the art that protect the skin from the cold in cryotherapy treatments and the like.

So, Chinese patent CN102440844 “CLINICAL PROTECTOR” is known, in the name of KAIYAN LIANG, from the year 2010, which relates to a clinical protector, which belongs to the field of medical apparatuses. The clinical protector comprises a circular guide ring, a first groove arranged on the upper side and a second groove arranged on the lower side in the circular guide ring, at least two outer horizontal deflectors fitted in the first groove, at least two inner horizontal deflectors fitted in the second groove, a vertical deflector fitted in the inner end parts of the inner horizontal deflectors, a protective rubber pad fitted in the vertical deflector, a first separation block and a second spacing block. The invention overcomes the defects of the known public technology, as the size of the clinical protector can be adjusted according to the shape of the coated part, so that this way the clinical protector is closely joined to the protected skin so that in turn it protects the healthy skin around the affected part of the patient and prevents the liquid nitrogen from being able to produce unnecessary damage on the healthy skin. The invention has the advantage of being a simple structure and low cost, it is comfortable to use, and considerably improves the safety of cryosurgery.

Also belonging to the state of the art is patent WO2007127924 “CRYOPROTECTOR FOR USE WITH A TREATMENT DEVICE FOR IMPROVED COOLING OF SUBCUTANEOUS LIPID-RICH CELLS”, from the year 2007, in the name of JUNIPER MEDICAL, INC, which relates to a cryoprotectant for use with a treatment device for improved removal of heat from subcutaneous lipid-rich cells of a subject having skin is provided. The cryoprotectant is a non-freezing liquid, gel, or paste for allowing pre-cooling of the treatment device below 0° C. while preventing the formation of ice thereon. The cryoprotectant may also prevent freezing of the treatment device to the skin or ice from forming from moisture seeping out from the skin.

It is also worth highlighting patent US2007/255362 “CRYOPROTECTANT FOR USE WITH A COOLING DEVICE FOR IMPROVED COOLING OF SUBCUTANEOUS LIPID-RICH CELLS”, from the year 2007, in the name of JUNIPER MEDICAL, INC, which relates to a cryoprotectant for use with a treatment device for improved removal of heat from subcutaneous lipid-rich cells of a subject having skin is provided. The cryoprotectant is a non-freezing liquid, gel, or paste for allowing pre-cooling of the treatment device below 0° C. while preventing the formation of ice thereon. The cryoprotectant may also prevent freezing of the treatment device to the skin or ice from forming from moisture seeping out from the skin. The cryoprotectant may further be hygroscopic, thermally conductive, and biocompatible.

So, patent US2008/0268216 “COOLING PRODUCT”, in the name of KIMBERLY CLARK CO, from the year 2007, which relates to a cooling product (e.g., pad, bandage, headband, wrap, cloth, and so forth) that contains a gel configured to cool the skin of a body part when placed adjacent thereto is provided. The gel is contained within a composite that has two or more fibrous layers structured to provide enhanced distribution of the gel therethrough. More specifically, a first fibrous layer may rapidly distribute the gel in primarily the −z direction (direction of thickness) to a second fibrous layer, which then distributes it primarily in the −x and −y directions. The second fibrous layer may then be placed adjacent to a user's skin to provide the desired cooling. Because the gel is distributed primarily in the −x and −y directions, however, direct contact between the gel and skin is limited, thereby reducing the “sticky” feel normally associate with such gels. The cooling product is also flexible and may be easily adapted to a body part.

Finally, the applicant firm is also owner of patent PCT/ES2017/070032, “SKIN AND TISSUE PROTECTION DEVICE FOR COLD AESTHETICS TREATMENTS”, from the year 2017, which relates to a skin and tissue protection device for cold aesthetics treatments, comprising at least a first layer of a cold-conducting material, the device being characterised in that it comprises: at least a second layer over the first layer and joined thereto, defining inner surfaces between both layers that form at least one housing, both layers being porous and absorbent; and at cold-conducting antifreeze gel spread in the housing, on the entirety of the inner surfaces, with a freezing point below −12° C. and with a viscosity between 2,000-90,000 cPs at 21° C., which is sufficient for passing through the pores of the layers and exiting the housing.

### BRIEF DESCRIPTION OF THE INVENTION

This application falls within the sector of the devices for cold aesthetics treatments and the so-called cryotherapy treatments.

The closest document is the aforementioned patent PCT/ES2017/070032.

Thus, said patent solves the problem of how to protect the dermal tissue from possibly freezing during the performance of cold treatments or cryotherapy.

Therefore, an anti-freezing gel is provided between the two layers of the device or membrane, so that the gel always impregnates the layers, preventing the skin from being damaged as a result of the ice, with the second layer, which is the one that is on the patient's skin, being made from a skin protection material.

Also, the actual double membrane adapts to the suction by the head piece while continuing to protect the skin.

The problem arises when some operators, due to lack of time or of experience, do not use the suction appropriately and do not place the membrane correctly and either spread the anti-freezing gel inside the double membrane before using it, and cause the membrane to break due to the suction action, or one part remains without the anti-freezing protector.

Therefore, the inventor has developed a new device, also called indistinctively membrane or double membrane, which improves the above in the aspect of protecting the patient from the problems mentioned above.

Also, it has the advantage that it can be used in head pieces of competitor's devices because the reinforcement guarantees that although there are sharp surfaces there is no risk of breaking the membrane, thereby making it possible to use it independently of the head piece manufacturer.

So, one of the layers comprises a stiffening structure that increases the stiffness of the device or membrane, thus avoiding that it breaks due to the suction action or the application of positive pressure (for example, when nitrogen is applied for cooling).

Therefore, the device's integrity is guaranteed as well as the even distribution of the anti-freezing gel in the housing, or housings.

An object of this invention is a skin and tissue protection device for cold aesthetic treatments, of the type comprising at least one first layer, at least one second layer on said first layer and joined to it, defining inner surfaces with both layers forming at least one housing, with both layers being porous and absorbent, and at least one cold conducting anti-freezing gel, spread in the housing, all over the inner surfaces, with a freezing point below −12° C., and with a viscosity between 2000 and 90000 cPs at 21° C., which is enough to pass through the pores of the layers and come out of the housing; characterized in that it comprises a reinforcement in at least one of its layers, such as a stiffening structure.

### BRIEF DESCRIPTION OF THE DRAWINGS

In order to facilitate the explanation, this specification includes eight sheets of drawings which represent a practical embodiment, which is shown as a non-limiting example of the scope of this invention:

FIG. 1 is a general view of some cold head pieces with the device of this invention,

FIG. 2 is a view of the head piece with the device and the skin,

FIG. 3 is a sectioned view of FIG. 2, along the line II-II, at the time of the cooling and suction of the device and the skin by the head piece,

FIG. 4 is a perspective view of the device,

FIG. 5 is a sectioned view along the line V-V of FIG. 4,

FIG. 5a is a detail of FIG. 5,

FIG. 6 is an exploded view of an embodiment where the reinforcements are in the second layer,

FIG. 7 is an optional sectioned view along the line V-V of FIG. 4,

FIG. 7a is a detail of FIG. 7,

FIG. 8 is an optional sectioned view along the line V-V of FIG. 4,

FIG. 8a is a detail of FIG. 8.

### SPECIFIC EMBODIMENT OF THIS INVENTION

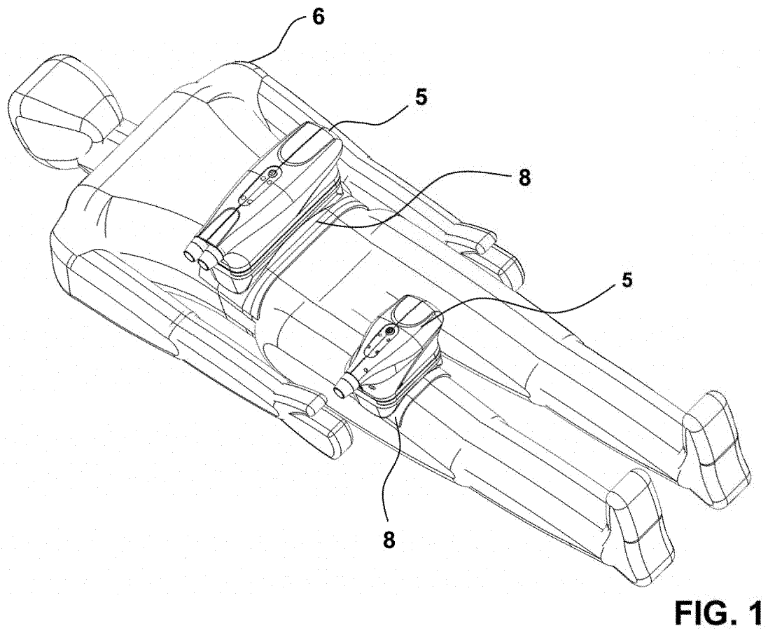

Thus, FIG. 1 illustrates a patient 6 on whom there have been placed some devices 8 that are the object of this invention with some head pieces 5 arranged on said devices 8.

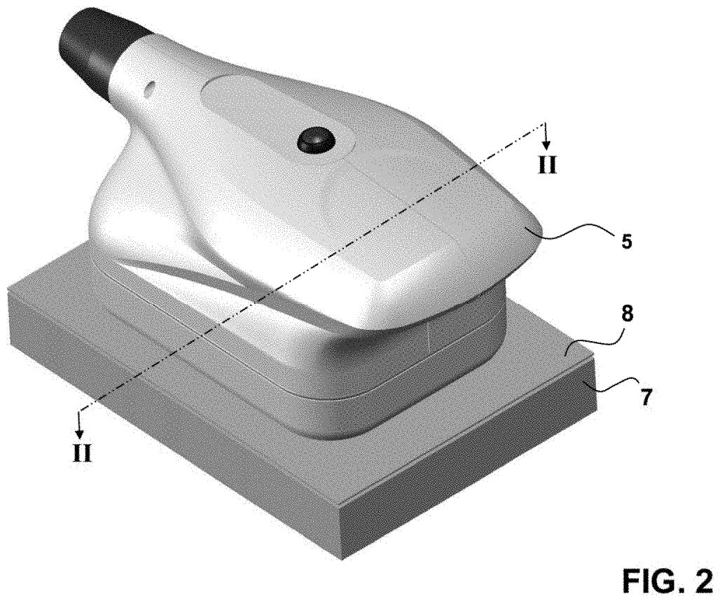

FIG. 2 represents head piece 5, device 8 and the patient's skin 7.

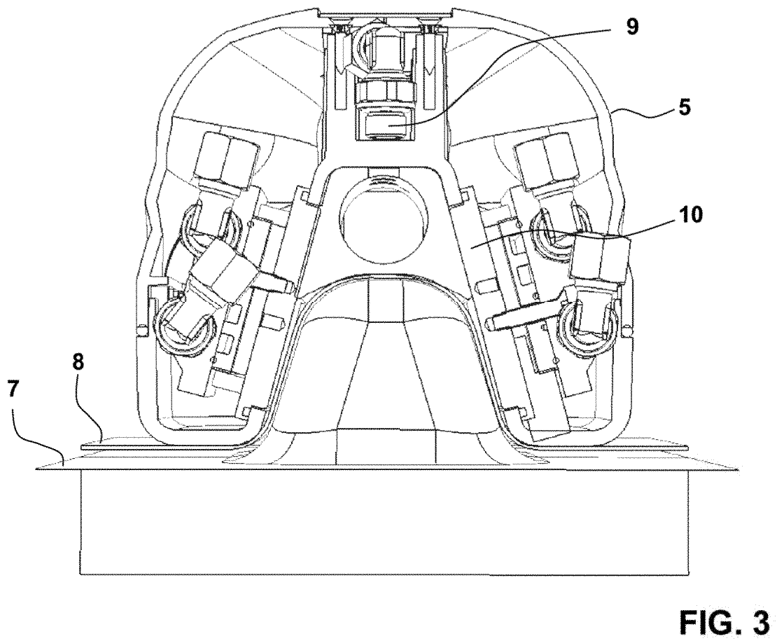

FIG. 3 shows the head piece with suction means 9, cold means 10, the device 8 and the patient's skin 7.

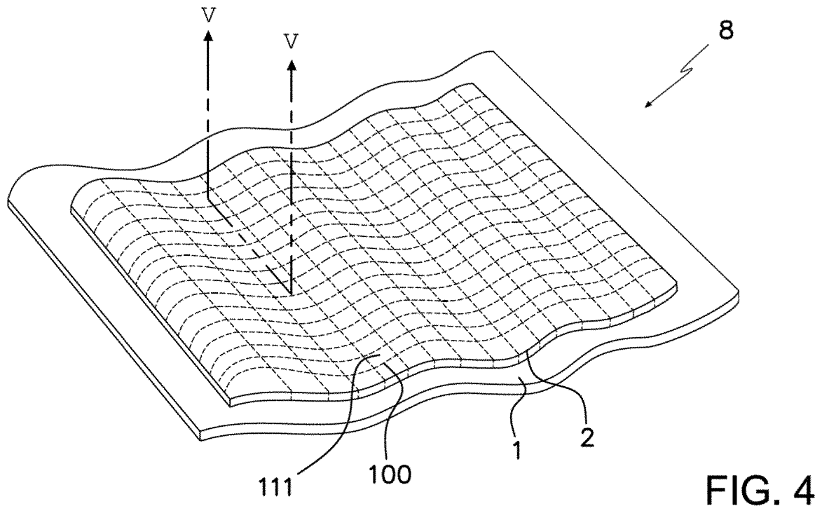

FIG. 4 illustrates device 8 with its first layer 1, its second layer 2, a reinforcement 100 and housings 111.

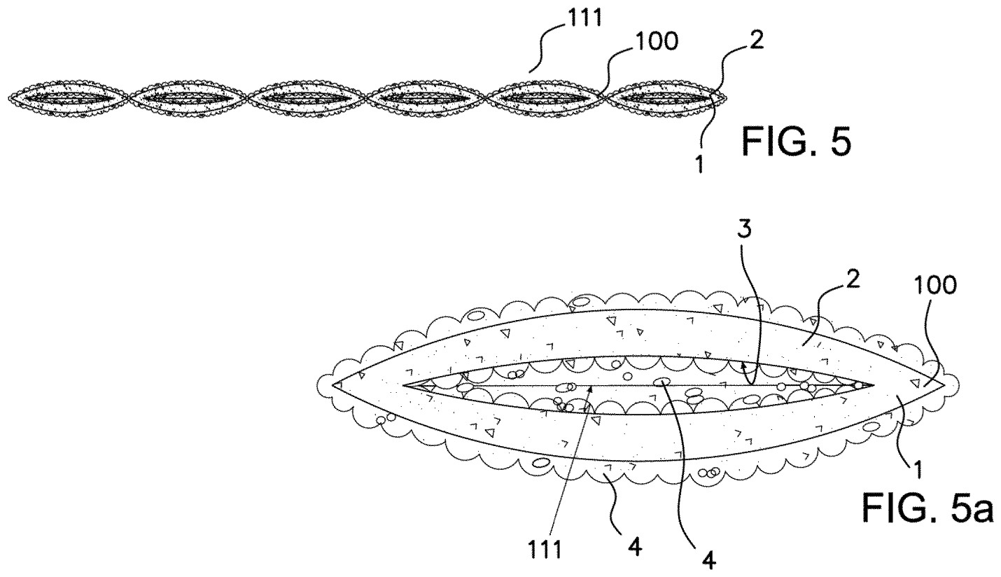

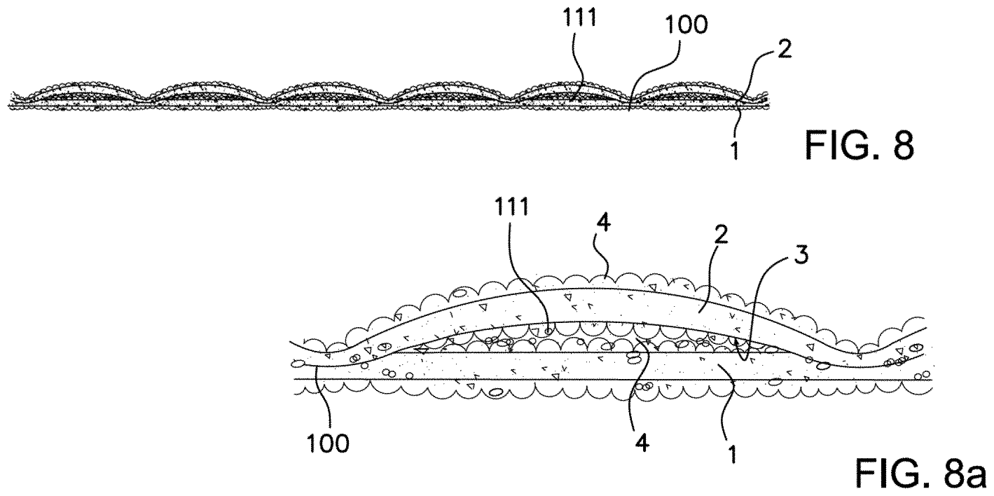

FIGS. 5, 7 and 8 represent the first layer 1, the second layer 2, housings 111 and the reinforcement 100.

FIGS. 5a, 7a and 8a illustrate the first layer 1, the second layer 2, inner surfaces 3, the housing 111, reinforcements 100 and an anti-freezing gel 4.

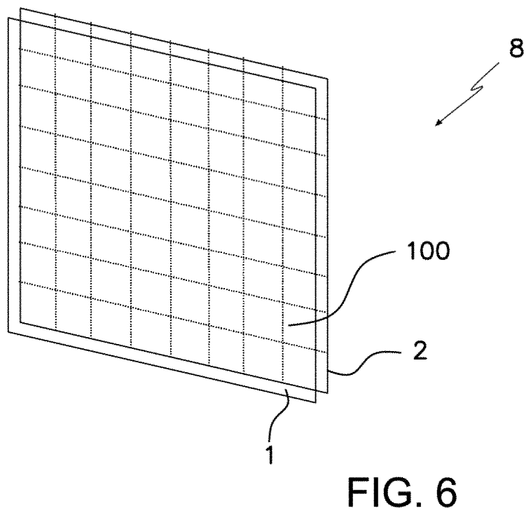

Finally, FIG. 6 shows the device 8, the first layer 1, the second layer 2 and reinforcements 100.

As a preliminary, FIGS. 5, 5a, 7, 7a, 8 and 8a show the anti-freezing gel 4. Usually as the first layer 1 is joined to the second layer 2 along its perimeter, it is not possible to see anti-freezing gel 4 from the outside, unless said anti-freezing gel 4 comes out through the porosities. Said figures show it in order to allow the reader to understand the position of anti-freezing gel 4 inside, even when in many of the embodiments it cannot be seen from outside.

Also, in FIGS. 5, 7 and 8, the same section cutting line V-V in FIG. 4 is used, because they are options within the same section cutting.

So, in a specific embodiment, the device 8, object of this invention, comprises at least one first layer 1 made of a porous material, which protects the patient's skin, and in this embodiment only one layer has been considered in principle, but one or several additional layers of hypoallergenic material, etc., could be added.

The material of first layer 1 has to be porous to allow anti-freezing gel 4, as the cold conducting medium, to transmit the cold to the anti-freezing gel 4 which is inside housing 111 or housings 111.

This way, the cold, through anti-freezing gel 4, ends up reaching the patient.

It comprises at least one second layer 2 made of porous material, on said first layer 1 and joined to it, forming a whole unit, defining inner surfaces 3 between said layers, in other words surfaces where both layers would be touching each other if gel 4 were not inside them. Between both inner surfaces a housing 111 is formed for storing the anti-freezing gel 4.

As with first layer 1, in this embodiment the second layer 2 only has one single layer, although it could have more than one if additional ones were arranged with hypoallergenic materials, creams, etc.

It also comprises at least one anti-freezing gel 4, with a freezing point below −12° C., spread on inner surfaces 3 inside the housing 111 leaving both layers 1,2 joined, and forming one single unit.

The temperature of the freezing point of anti-freezing gel 4 must be equal to or lower than −12° C., since even when the working temperature is usually −8° C., there are times when due to the type of treatment to be carried out the temperature can be −12° C., and tests have even been carried out that have reached up to −20° C.

Said layer must be porous in order to be able to transmit the cold to the patient's skin 7 through anti-freezing gel 4.

In other words, the anti-freezing gel 4, which is in contact with the cold that is given off by the cold means 10 in the head piece 5, transmits the cold to anti-freezing gel 4, which is in the housing 111, through the porosities in the first layer, and the cold that gel 4 has received from housing 111, in turn transmits the cold to anti-freezing gel 4 which is in contact with the patient's skin through the porosities of second layer 2 (FIGS. 5, 7 and 8).

With respect to anti-freezing gel 4, this must have sufficient viscosity, and it has been calculated that it should be between 2000 and 90000 centipoises (cPs) at 21°, Rotor 3 at 3 RPM, in order to pass through the pores of the layers 1,2 and leave the housing 111. An intermediate, standard viscosity could be between 35000 and 38000 cPs.

An example of the composition of anti-freezing gel 4 could be:
Solvent, for example, water, between 25 and 50%,
Solvent-wetting agent, for example, glycerine, between 50 and 75%,
Viscosity controllers (for example, cellulose), between 0.1 and 2.0%, and
Preservatives (for example, magnesium nitrate, magnesium chloride, etc., between 0.01 and 0.1%).

Moreover, said anti-freezing gel 4 of the example must have the following at 21° C.:
A pH between 5.0 and 7.0 pH units, and
A density between 0.990 and 1.200 g/ml.

The visual perception must be stable in all assays.

The indicated margins depend on the porosity of first layer 1 and second layer 2, if they are more porous, in other words, let more anti-freezing gel 4 pass through, the viscosity will reduce and it will flow more slowly between the pores in layers 1, 2, coming close to 90000 cPs, while on the other hand, if layers 1,2 are less porous, the viscosity of anti-freezing gel 4 will augment to increase the fluidity of anti-freezing gel 4 between the pores in layers 1,2, coming close to 2000 cPs.

In order to increase the efficiency of the anti-freezing gel 4, it has been considered to distribute said anti-freezing gel 4 evenly on all inner surfaces 3. This is done because it prevents risks of burning in the event that one part of device 8 were without any gel and were being suctioned and cooled by head piece 5. Also, it could be possible to use the embodiment explained later, and shown in FIGS. 5, 7 and 8, of different individual housings 111, which allow the amount of anti-freezing gel 4 to be retained in a specific area due to the action of housings 111.

If it happened that one part of device 8 were facing one part of the patient's skin without anti-freezing gel 4, ice would be produced, and the consequent burn.

Therefore, in the event that some anti-freezing gel 4 were missing in one part of device 8, it would allow the cold and suction to be directed against the patient's skin 7, causing damage and burns on said patient's skin 7.

It could also be possible that first layer 1 and second layer 2 are a different size, and preferably that the smaller-sized layer is placed in the centre of the larger-sized layer.

Preferably, the smallest one is first layer 1, and it should be big enough to cover the whole of the inside of head piece 5 when it produces the suction action, and this way, as there will be anti-freezing gel 4 covering the whole of the area of said first layer 1, there is no risk of there being any areas on the patient's skin 7 where cold and suction are applied, that do not have anti-freezing gel 4.

Optionally, layers 1,2 are joined together either by sewing, heat welding or sticking, although this list is not limited.

Among the porous materials from which layers 1,2 can be made, cellulose could be used. The grammage or weight of the cellulose could be in the order of between 10 and 200 g/cm³.

Optionally, layers 1,2 could have a different weight, as the greatest weight is of interest in first layer 1 which is the one that withstands the tensions of suction means 9, with it being foreseen that pressures of up to 500 mbar can be withstood without the fibres breaking.

Due to manufacturing issues, it could be of interest that second layer 2 had the same weight as first layer 1, as this way the whole device 8 could be made with one single cellulose.

Optionally, other materials can also be used instead of the cellulose, such as paper, natural or synthetic fibres that are included in the polyester group, synthetic polymers such as nylon, or also teflon.

As for using paper as a porous material, this could have a pore size between 2 and 30 micrometres and a weight between 10 and 200 g/m².

In the case of the synthetic polymers you could choose nylon as a porous material with a pore size between 0.1 and 180 micrometres.

With respect to the option of using teflon (polytetrafluoroethylene PTFE), its size could be between 0.1 and 10 micrometres.

As shown in FIG. 4, the device 8 or the membrane that is the object of this invention comprises a reinforcement 100. In this embodiment it has been shaped as a mesh, even though it could adopt another more convenient shape according to the head piece to be used, such as in concentric circles, etc.

Reinforcement 100 is placed in one of layers 1,2, in this embodiment in the second one 2, since this way it is the one that supports all the suction from the suction means 9 of head piece 5.

Also, reinforcement 100 guarantees that even when it is used in head pieces 5 of the competitor's devices, although there may be sharp edges or surfaces, device 8 is not going to break.

The reinforcement 100 can be formed directly on one of the layers (FIG. 6), for example, on the second layer 2, which is in contact with suction means 9, or said reinforcement 100 joins the second layer 2 to the first layer 1, defining various housings 111 where an amount of anti-freezing gel 4 is retained, as shown in FIGS. 5, 5a, 7, 7a, 8 and 8a.

Also, reinforcement 100 could be formed on both layers 1,2, as, for example, shown in FIG. 5.

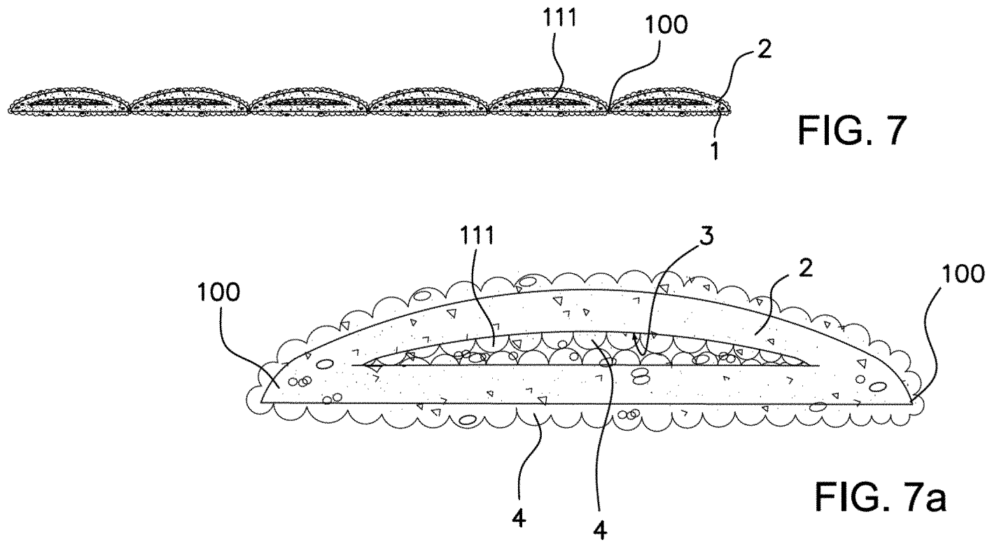

If various housings 111 are formed, as indicated in FIGS. 5 and 7, this allows a more even distribution of anti-freezing gel 4, avoiding the risk of anti-freezing gel 4 escaping longitudinally, by establishing a distribution of pressures between skin 7 and head piece 5.

One of the advantages of this configuration is that taking into account that the gel moves longitudinally within housings 111, said housings 111 define a volume that does not move to other parts of device 8, which means that the anti-freezing effect of gel 4 is guaranteed.

The reinforcement 100 can be made by sewing, gluing, heat welding, etc., and the list is not being exhaustive or non-limiting. For example, synthetic or natural fibres could be sewn or a polyester for sewed reinforcement 100. Other options could be cotton threads, inserting strips of polymeric material or lines of silicone that would provide stiffness.

When a cryotherapy treatment is going to be carried out on a patient 6, a device 8 would be placed on the patient's skin 7.

The second layer 2 would be on the patient's skin 7 and first layer 1 would be opposite head piece 5 (as an example we can use the one described in EP2687174, by the actual applicant firm).

Next, the opening of head piece 5 would be placed on the first layer 1 and suctions means 9 and cold means 10 would be started.

This would lead to a situation such as the one in FIG. 3 where one part of the patient's skin 7 is absorbed by suction means 9, but leaving device 8 between said means, that protects the patient's skin 7 from the action mainly from the cold means, which avoids ice forming on the patient's skin.

This way, the cold can penetrate under the patient's skin 7, transmitted by anti-freezing gel 4 through the porosity of second layer 2; but on the other hand, anti-freezing gel 4 prevents ice from forming on the patient's skin 7 without causing injuries, and without causing burns.

The reinforcement 100 guarantees safety, as it prevents device 8 from breaking, by removing the consequent danger of the user's skin burning.

Also, it guarantees that the anti-freezing gel 4 is well distributed inside the device and with a volume of gel in each housing. If a device such as the ones in FIGS. 5, 7 and 8 is used, anti-freezing gel 4 has less chance of moving inside device 8 and therefore it is better distributed as it does not move inside housings 111 or cells, and therefore it cannot move inside device 8, but only inside its own housing 111 or cell, which guarantees the safety of the device 8 because it reduces the risk of lack of the anti-freezing gel 4 inside housings 111 or cells, because the reinforcement 100 limits the movement of the gel, by confining it partially inside each housing 111 or cell, which means that the gel can no longer move to other areas of device 8.

This invention describes a new skin and tissue protection device for cold aesthetics treatments. The examples mentioned herein are non-limiting of this invention, and therefore can have different applications and/or adaptations, all within the scope of the following claims.

1. A skin and tissue protection device for cold aesthetics treatments, of the type comprising:
at least a first layer (1),
at least a second layer (2) on said first layer (1) and joined to it (1), defining inner surfaces (3) between both layers which form at least one housing (111), with both layers (1, 2) being porous and absorbent, and
at least one cold conducting anti-freezing gel (4), spread in housing (111), all over inner surfaces (3), with a freezing point below −12° C., and with a viscosity of between 2000 and 90000 cPs at 21° C., which is enough to pass through the pores in layers (1, 2) and come out of housing (111)
characterized in that it comprises a reinforcement (100) in at least one of its layers (1, 2), such as a stiffening structure.

2. A device, according to claim 1, characterized in that the reinforcement (100) is placed in second layer (2).

3. A device, according to claim 1 or 2, characterized in that the reinforcement (100) joins the first layer (1) to second layer (2) defining cells (111) inside which anti-freezing gel (4) is housed.

4. A device, according to claim 3, characterized in that the reinforcement (100) has been sewn, heat welded or glued.

5. A device, according to claim 3, characterized in that the first layer (1) and the second layer (2) are the same size.

6. A device, according to claim 3, characterized in that the first layer (1) and the second layer (2) have a different size.

7. A device, according to claim 1 or 2, characterized in that the material of layers (1, 2) is cellulose.

8. A device, according to claim 7, characterized in that the weight of the cellulose is between 10 and 200 g/cm3.

9. A device, according to claim 8, characterized in that layers (1, 2) have a different weight.

10. A device, according to claim 8, characterized in that layers (1, 2) have the same weight.

11. A device, according to claim 1 or 2, characterized in that the material of layers (1,2) is paper.

12. A device, according to claim 1 or 2, characterized in that the material of layers (1,2) are natural or synthetic fibres that are included in the polyester group.

13. A device, according to claim 1 or 2, characterized in that the material of layers (1,2) are synthetic polymers such as nylon.

14. A device, according to claim 1 or 2, characterized in that the material of layers (1,2) is teflon.
